SEQUENCE LISTING (19 sequences)

SEQ ID NO: 1 - DNA, 950 bp, Artificial Sequence
  ColE1 plasmid
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccaatcgatcccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 2 - DNA, 950 bp, Artificial Sequence
  ColE1 plasmid
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccaatcgatcccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacctcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgccaggggggcggagcctatggaaaaacgccagcaacgcggcctttttacggttcctggccttttgctggccttttgctcacatgt

SEQ ID NO: 3 - DNA, 950 bp, Artificial Sequence
  ColE1 plasmid
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccaatcgatcccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctttgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcttggccttttgctggccttttgctcacatgt

SEQ ID NO: 4 - DNA, 950 bp, Artificial Sequence
  ColE1 plasmid
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccaatcgatcccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgctcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataagacgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggacggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 5 - DNA, 950 bp, Artificial Sequence
  ColE1 plasmid
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccaatcgatcccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagagggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 6 - DNA, 950 bp, Artificial Sequence
  ColE1 plasmid
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccaatcgatcccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgtagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaagggggcggagcctatggaaaaacgccagcaacgcggcctttttacggttcctggccttttgctggccttttgctcacatgt

SEQ ID NO: 7 - DNA, 950 bp, Artificial Sequence
  ColE1 plasmid
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccaatcgatcccgtagaaaagatcaaaggatcttcttgagatcctttttttctgcgcgtaatctactgcttgcaaacaaaaaaaccaccgctaccagcggtggtttttttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttgaagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 8 - DNA, 950 bp, Artificial Sequence
  ColE1 plasmid
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccaatcgatcccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtatagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 9 - DNA, 950 bp, Artificial Sequence
  ColE1 plasmid
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccaatcgatcccgtagaaaagatcaaaggatcttcttgagatcctttttttctgcacgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtagctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 10 - DNA, 950 bp, Artificial Sequence
  ColE1 plasmid
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccaatcgatcccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctttgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 11 - DNA, 950 bp, Artificial Sequence
  ColE1 plasmid
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccaatcgatcccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtagctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 12 - DNA, 950 bp, Artificial Sequence
  ColE1 plasmid
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccaatcgatcccgtagaaaagatcaaaggatcttcttgagatcctttttttctgcgcgtaatctactgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccatcacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagagtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 13 - DNA, 37 bp, Artificial Sequence
  ColE1 plasmid
cttcttgagatcctttttttctgcgcgtaatctgctg

SEQ ID NO: 14 - DNA, 28 bp, Artificial Sequence
  ColE1 plasmid
ggatcaagagctaccaactctttttccg

SEQ ID NO: 15 - DNA, 36 bp, Artificial Sequence
  ColE1 plasmid
cttctagtgtagccgtagttaggccaccacttcaag

SEQ ID NO: 16 - DNA, 41 bp, Artificial Sequence
  ColE1 plasmid
atactgttcttctagtgtagccgtagttaggccaccacttc

SEQ ID NO: 17 - DNA, 22 bp, Artificial Sequence
  ColE1 plasmid
tcgctctgctaatcctgttacc

SEQ ID NO: 18 - DNA, 15 bp, Artificial Sequence
  ColE1 plasmid
agggggaaacgcctg

SEQ ID NO: 19 - DNA, 9 bp, Artificial Sequence
  ColE1 plasmid
ggggggcgg

(19) United States
(12) Patent Application Publication
(10) Pub. No.: US 2013/0266983 A1
(43) Pub. Date: Oct. 10, 2013

(54) Creation of super-stable ColE1 plasmids by duplication of SL1-4 sequence and point mutations.

(71) Applicants: Manel Camps, Santa Cruz, CA (US); Samuel Million-Weaver, Santa Cruz,, CA (US)

(72) Inventors: Manel Camps, Santa Cruz, CA (US); Samuel Million-Weaver, Santa Cruz,, CA (US)

(73) Assignee: The Regents of The University of California, Oakland, CA (US)

(21) Appl. No.: 13/990,510

(22) PCT Filed: Dec. 14, 2011
(86) PCT No.: PCT/US2011/064982

§ 371 Date: May 30, 2013

Related U.S. Application Data
(60) Provisional application No. 61/422,710, filed on Dec. 14, 2010.

(51) Int. Cl. C12N 15/68

(52) U.S. Cl. CPC C12N 15/68
USPC 435/69.1; 435/320.1

(57) ABSTRACT

Super-stable plasmids useful to improve yields of large-scale recombinant gene expression.

### RELATIONSHIP TO OTHER APPLICATIONS

This application claims the benefit of an priority to U.S. provisional application No. 61/422,710 titled “Duplication and drift of SL1-4 sequence in ColE1 plasmid origins of replication leads to a dramatic enhancement in plasmid stability”, filed on 14 Dec. 2010, which is hereby incorporated by reference for all purposes.

### STATEMENT OF SUPPORT

This invention was made with government support under the following grants: K08-CA116429-04. The government has certain rights in the invention.

### FIELD OF THE INVENTION

Engineered super-stable plasmids useful for increasing yield of commercial processes for manufacturing high value proteins and other products.

### BACKGROUND

ColE1 plasmids are the most widely used vectors for recombinant gene expression. ColE1 plasmid replication is orchestrated by a −550 bp sequence known as plasmid origin of replication (ori) whose transcript serves as a primer for leader strand synthesis by DNA polymerase I (FIG. 1a). Plasmid replication initiation is regulated by a negative feedback loop mediated by four stem-loop structures (SL1-4). Hybridization of three of these stem-loops (SL1-3) with an antisense RNA transcript blocks replication initiation. Since the levels of antisense RNA are proportional to plasmid copy number, this mechanism constitutes a negative feedback loop that maintains a constant plasmid copy number for a given culture and culture condition. Further elongation of the transcript leads to the formation of the fourth loop. This additional loop (SL4) makes the pre-primer RNA refractory to the antisense inhibitor RNA restricting negative feedback action to nascent transcripts. In the absence of a functional selection, however, plasmids are rapidly lost from the population. Plasmid loss associated with recombinant gene expression constitutes one of the main factors limiting large-scale production of DNA, proteins, and secondary metabolites.

### BRIEF DESCRIPTION OF THE INVENTION

Here we present plasmids having an engineered, mutant ColE1 ori whose replication is stable over multiple generations in the absence of any genetic or chemical selection. This increased stability is associated with tighter copy number regulation, likely provided by a duplication in the antisense RNA circuitry and assisted by point mutations. The resulting decrease in plasmid copy number variability limits the frequency of random, spontaneous plasmid loss, substantially prolonging plasmid retention. Clearly this provides very substantial commercial advantages in terms of increased productivity and decreased cost of production and reduced use of selective antibiotics.

ColE1 plasmids are the most widely used vectors for recombinant gene expression but are rapidly lost in the absence of selection. Under large-scale (industrial) conditions, selections are impractical. Therefore, plasmid stability is one of the main factors determining the yield of large-scale recombinant protein or secondary metabolite production.

Here we present a plasmid having an origin of replication (ori) sequence that confers stable ColE1 plasmid replication in E. coli cells. Our engineered ori is a hybrid containing a duplicated segment of stem-loops 1-4 (SL1-4; secondary structures critical for copy number regulation) and point mutations modulating plasmid copy number. These constructs show some (1-2.5 fold) increase in copy number and a dramatic increase in stability, with up to 1% cells retaining plasmid following 13 passages (200 generations) of growth in the absence of selection. Thus, our modified ori allows stable replication of plasmid-encoded genes for multiple generations without the need for selection and functional complementation. This should greatly facilitate large-scale production of DNA, proteins, and secondary metabolites. The observed increase in plasmid stability is likely attributable to the presence of two functional antisense negative feedback loops, tightening regulation. Decreased plasmid copy number variability across the population would reduce the frequency of random plasmid loss, favoring plasmid stability.

In the present invention, the generation of ColE1 plasmid origins of replication containing duplicated antisense feedback loop sequence and additional point mutations provides high stability in the absence of drug selection. Super-stable plasmids show a narrowed range of copy numbers, suggesting that copy number distribution is a key determinant of plasmid stability and that it is susceptible to genetic modulation. Our engineered plasmids significantly improve yields of large-scale recombinant gene expression.

Engineered ori sequences involve a duplication at the 5′ end of ori encompassing SL1-4 (the key secondary structures mediating antisense RNA regulation of replication initiation) and modulating point mutations. The duplication of the SL1-4 copies creates what we believe to be a second functional negative feedback loop, tightening up plasmid copy number regulation, and decreasing the probability of random, spontaneous plasmid loss. Additional point mutations significantly enhance high stability.

Various non-exclusive embodiments of the invention include the following.

A plasmid comprising an engineered ColE1 (or other expression vector) sequence which when transformed into a host cell exhibits levels of plasmid retention at least 100 times that of a wild type control under the same conditions following 13 passages (200 generations). In other embodiments plasmid retention is at least 10³, 10⁴, 10⁶ or 10⁶ times greater than wild type.

An engineered, mutant ColE1 ori plasmid (or other expression vector) whose replication is stable over multiple generations in the absence of any genetic or chemical selection wherein the plasmid comprises a Col E1 origin of replication containing a duplicated antisense feedback loop sequence. It may also possess at least one additional stabilizing point mutation.

A method for production of polynucleotides, proteins, glycoproteins, or secondary metabolites, the method comprising culturing a cell transformed with an engineered plasmid (such as one described herein) providing stable replication of plasmid-encoded genes for multiple generations without the need for selection and/or functional complementation. This method may employ an engineered plasmid wherein the plasmid comprises a Col E1 (or other expression vector) origin of replication containing a duplicated antisense feedback loop sequence. It may also have at least one additional stabilizing point mutation.

A plasmid whose replication is stable over multiple generations in the absence of any genetic or chemical selection, comprising an engineered ColE1 (or other expression vector) sequence, wherein the ColE1 sequence includes at least one origin of replication comprising one or more duplicated sequences selected from the group consisting of:
(a) a duplication of the antisense feedback loops 1, 2, and 3 having at least 80% sequence identity with the wild type sequence,
AAAAAACCACCGCTACCAGCGGTGGTTTGCTTTGCCGGATCAAGAGCTACCAACTC=CCGAAGG TAACTGGCTTCAGCAGAG,
(b) a duplication of the P2 promoter sequence having at least 80% sequence identity with the wild type sequence,
TCTTGAGATCCTTTITTTCMCGCGTAATCTGCTGCT, and
(c) a duplication of the Stem-Loop 4 sequence having at least 80% sequence identity with the wild type sequence,
AAATACTGTTCTTCTAGTGTAGCCGTAGTTAGGCCACCACTTCA
(d) a duplication of the P1 antisense RNA promoter sequence having at least 80% sequence identity with the wild type sequence,
ACCAAATACTGTTCTTCTAGTGTAGCCGTAGTTAGGCCACCACT
(e) a duplication of the beta-stem sequence having at least 80% sequence identity with the wild type sequence TCGCTCTGCTAATCCTGTTACC.

The plasmid described above wherein the named duplicated sequence(s) have at least 60% or 70% or 80% or 90% or 95% or 99% sequence identity with the wild type sequence, or in some cases 100% sequence identity.

The plasmid described above comprising a duplication of the entire 5′ terminal 269 nucleotides of the ori sequence,
GTAGAAAAGATCAAAGGATCTICTTGAGATCCTTTTTTTCTGCGCGTAATCTGCTGCTTGCAAACAA AAAAACCACCGCTACCAGCGGTGGTTTGTTTG CCGGATCAAGAGCTACCAACTTTCCGAAGGTAACTGGCTTCAGCAGAGCGCAGATACCAAAT ACTGTTCTTCTAGTGTAGCCGTAGTTAGGCCAC CACTTCAAGAACTCTGTAGCACCGCCTACATACCTCGCTCTGCTAATCCTCTTACCAGTGGCTGCTGC CA. This duplication may be a 100% identical duplication over the entire sequence shown here, or may be truncated at either end to provide a duplication with, for example at least 60% or 70% or 80% or 90% or 95% or 99% sequence identity with the sequence shown.

The plasmid described above additionally comprising at least one stabilizing point mutation of a nucleotide residue selected from the group consisting of:
Position 44 in the P1 primer promoter region, Position 53 in the P1 primer promoter region, Position 95 in the SL1 region, Position 113 in the SL2 region, Position 116 in the SL2 region
Position 170 in the antisense promoter region, Position 180 in the antisense promoter (overlapping with SL4 region), Position 189 in the antisense promoter (overlapping with SL4 region), Position 198 in the antisense promoter (overlapping with SL4 region), Position 202 in the antisense promoter (overlapping with SL4 region)
Position 238 in the beta-stem region, Position 512 in the Hairpin 2 region, Position 592 in the C-rich region, within area of displaced strand, Position 592 in the displaced strand region, Position 325 in the Intervening sequence region, Position 328 in the Intervening sequence region, Position 338 in the Intervening sequence region
Position 373 in the Intervening sequence region, Position 409 in the Intervening sequence region, Position 477 in the Intervening sequence region, Position 479 in the Intervening sequence region, Position 589 in the Intervening sequence region, Position 643 in the DNA extension region, and Position 646 in the DNA extension region. Often there will be only one stabilizing point mutation but in other embodiments there may be more than one for example at least 2, 3, 4, 5, or at least 6 stabilizing point mutations.

A specific embodiment having one or more point mutations includes the plasmid described above additionally comprising the three point mutation(s) shown in DAS-C170T G325A and T479A : C to T at nucleotide position 170, G to A at nucleotide position 325, an T to A at position 479. Or in another embodiment the plasmid described above additionally comprising the two point mutation(s) shown in DAS- DAS-G44A G258A: G to A mutation at nucleotide position 44, and G to A at nucleotide position 258. These two mutants are particularly stable and exhibit close to 1% plasmid retention following ˜200 generations of passage in the absence of selection. Thus, these two mutant oris allow stable replication of plasmid-encoded genes for multiple generations without the need for selection or functional complementation. This should greatly facilitate large-scale production of DNA, proteins, and secondary metabolites.

Certain embodiments comprise point mutation(s) in an region selected from the group consisting of: SL1, SL2, SL4, antisense promoter, β stem, Hairpin 2, the C-rich region, the region defined by nucleotides 325-328, and the region defined by nucleotides 477-479.

Another embodiment of the invention encompasses a method for controlling plasmid copy number by modulation of plasmid copy number variance within a population. This is a potentially very important commercial embodiment. The rationale stems from the theory that the subpopulation of cells with no plasmid is the one driving plasmid loss. Therefore, controlling the variance of plasmid copy within a population is much more important than controlling the average copy number. The plasmids disclosed herein are much more stable with only modest increase in copy number and their stability does not correlate with copy number. This observation agrees with this theory and our FACS data confirms it. It is believed that this is the first time anyone has been able to demonstrate that modulation of plasmid copy number variance can have such a profound effect on plasmid stability. This is in contrast to other approaches for optimization of plasmid-driven recombinant gene expression such as: Minimizing metabolic burden, Increasing copy number, and Induction of quiescence.

Modulation of plasmid copy number variance within a population may be achieved genetically, as thoroughly disclosed herein by duplication of various regions of ori, and/or point mutations, but it may also be achieved by subjecting the population to other conditions known to influence copy number such as temperature, saturation of the culture, availability of amino acids, presence of additional plasmids, level of expression of recombinant genes etc.

Thus we also claim a method for controlling plasmid copy number by modulation of plasmid copy number variance within a population. Additionally we claim such a method employing one or more the following: (a) genetically, by duplication of various regions of ori, and/or point mutations, (b) by subjecting the population to increased temperature, (c) by saturation of the culture, (d) by altering availability of amino acids, (e) by presence of additional plasmids, (f) by altering the level of expression of recombinant genes.

It is important to note that although ColE1 plasmids are used as examples, many other plasmids and expression vectors may equally be used in the invention and there are many vectors widely used for recombinant gene expression that may equally be manipulated to provide a high stability plasmid by duplication of various regions including those found in the origin of replication region, and optionally may also possess at least one stabilizing point mutation. Duplicated regions may include (a) a duplication of the antisense feedback loop having at least 80% sequence identity with the wild type sequence, (b) a duplication of a promoter sequence having at least 80% sequence identity with the wild type sequence, (c) a duplication of a Stem-Loop sequence having at least 80% sequence identity with the wild type sequence, (d) a duplication of an antisense RNA promoter sequence having at least 80% sequence identity with the wild type sequence, and/or (e) a duplication of a beta-stem sequence having at least 80% sequence identity with the wild type sequence. Expression vectors of the invention may include vectors used commercially and in research including vectors used in prokaryotic and eukaryotic systems available from such companies as Promega, and Invitrogen.

### BRIEF DESCRIPTION OF THE FIGURES

Table 1(a) and (b). Monitoring plasmid loss. (a) Copy number analysis in individual cells by flow cytometry. Listed is the % of cells with no plasmid (NP) and with low plasmid (LP) following different passages in the absence of drug selection, each passage representing a 1:10⁵ dilution. (b) Plasmid retention. Listed is the number of cells from a culture of 1×10⁹ viable cells that retain at least one plasmid. Flow cytometry and marker retention experiments were performed independently. Their methods are described in Supplementary Methods.

FIG. 1(a)-(e). Plasmid copy number population dynamics. Cells grown either in the presence or in the absence of drug were prepared and analyzed by flow cytometry (a-d) or for marker retention (e) as described in Supplementary Methods. a-d Flow Cytometry. Cells transformed with WT pGFPuv (a), DAS (b), DAS-G44A G258A (c), and DAS-T170C G325A T479A (d) were grown in the presence of drug (black lines) or in its absence (light grey lines) and analyzed for fluorescence emission. The percentage of cells in the “no plasmid” and “low-plasmid” copy number brackets are indicated. e Marker retention of selected clones over increasing number of passages in the absence of drug, as listed for in Table 1b.

FIG. 1(a)-(e): x axis=fluorescence intensity at 531 nm, and y axis=number of cells.

FIG. 2: A table showing functional areas, position and stabilizing mutations

FIG. 3: Plasmid ori position vs Distance to next mutation

FIG. 4 Number of mutations vs Distance to next mutation in 5 ny intervals. Supplementary table 1: map of stabilizing point mutations. Positively-selected plasmid ori positions are listed, grouped by functional feature, in column 3. The nucleotide positions spanning each functional domain are listed in column 2, according to standard ColE1 numbering. Mutant positions corresponding to the two most stable clones are highlighted in bold, and mutant positions that appear as singles are underlined.

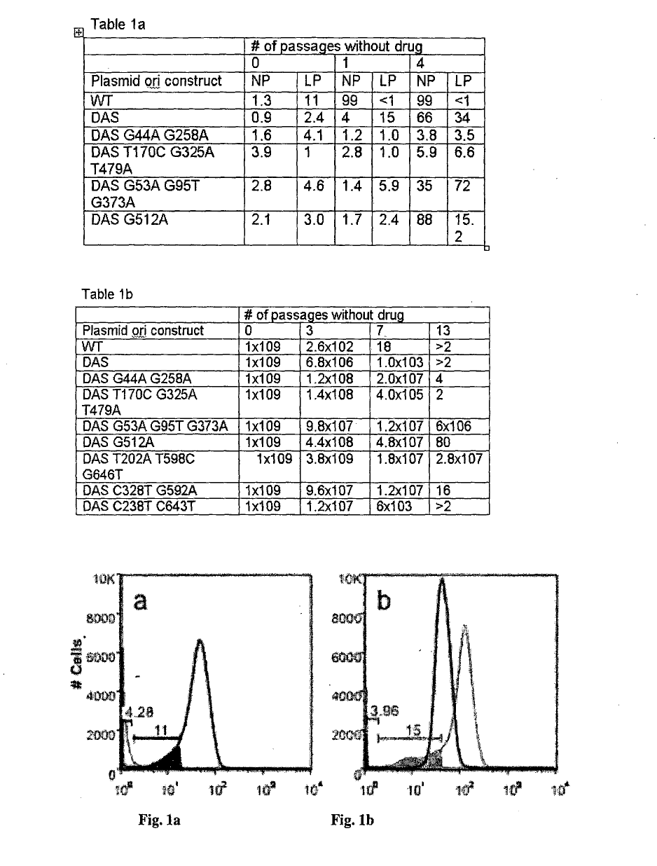

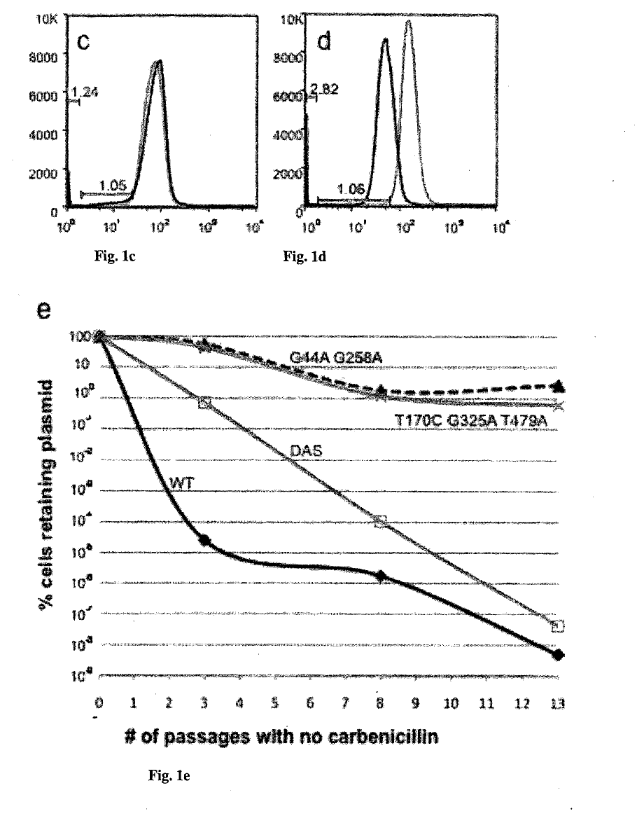

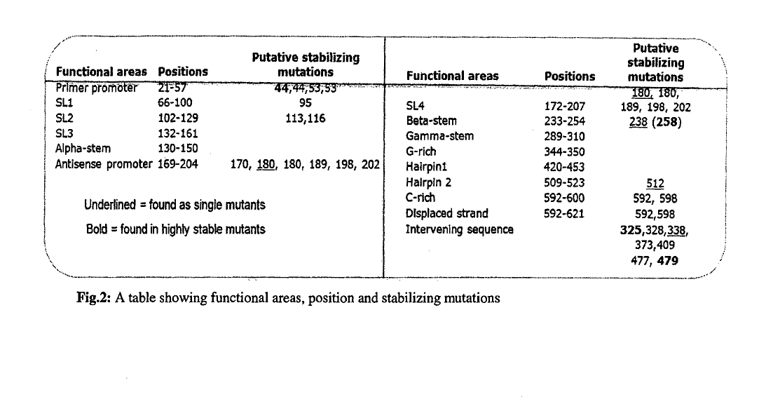

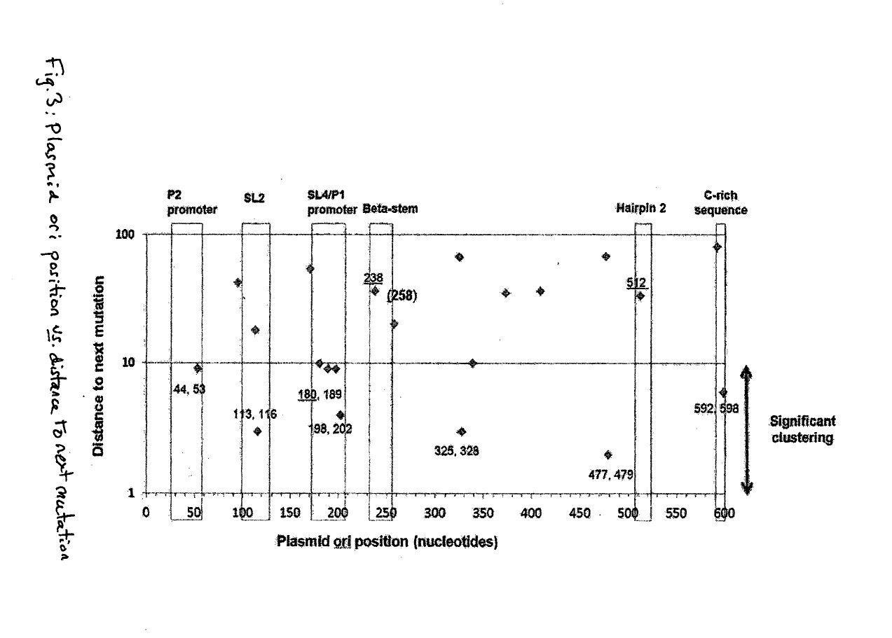

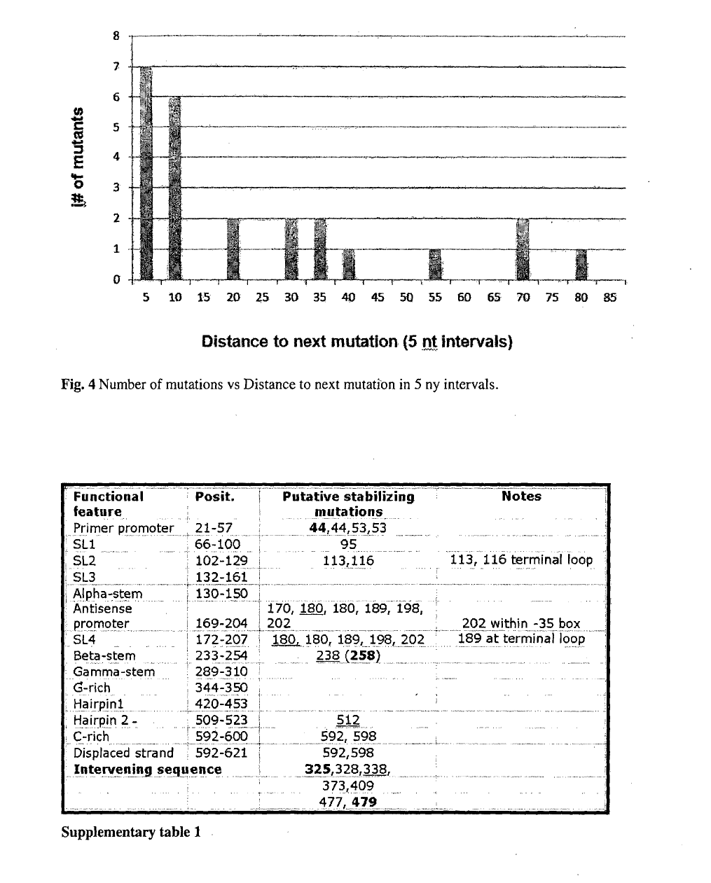

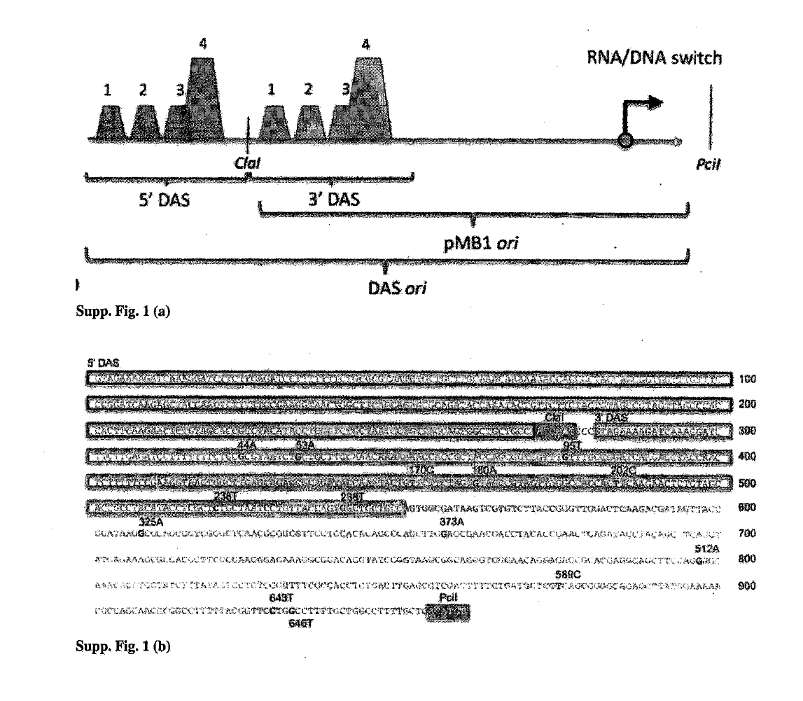

Supp. FIG. 1: (a) A Schematic showing the duplication of antisense regulatory sequence elements located at the 5′ end of the pMB1 (ColE1-like) ori of pGFPuv. The schematic also shows stem-loop structures that are important for antisense regulation, the two restriction sites used for construction, and the position of the RNA/DNA switch.

Supp. FIG. 1: (b) Sequence of DAS mi. Duplicated sequences are boxed; mutations tested for increased stability are highlighted in bold, denoting nucleotide positions (relative to the start of the ori following standard ColE1 numbering) and base pair substitutions.

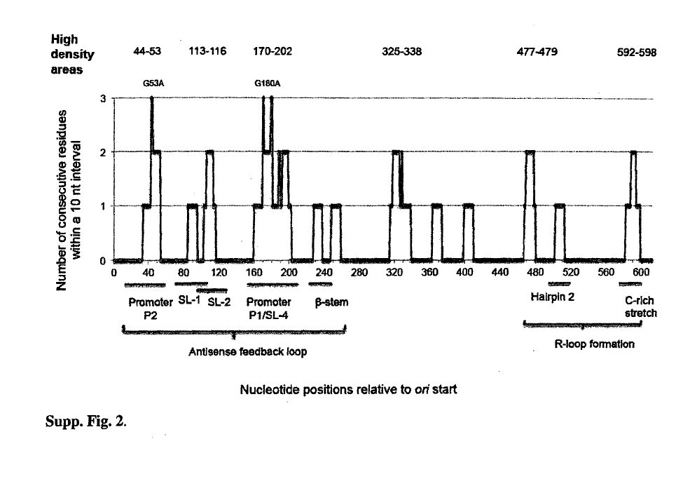

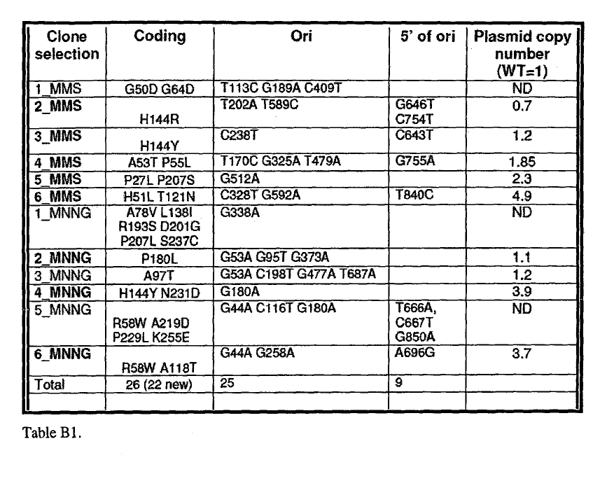

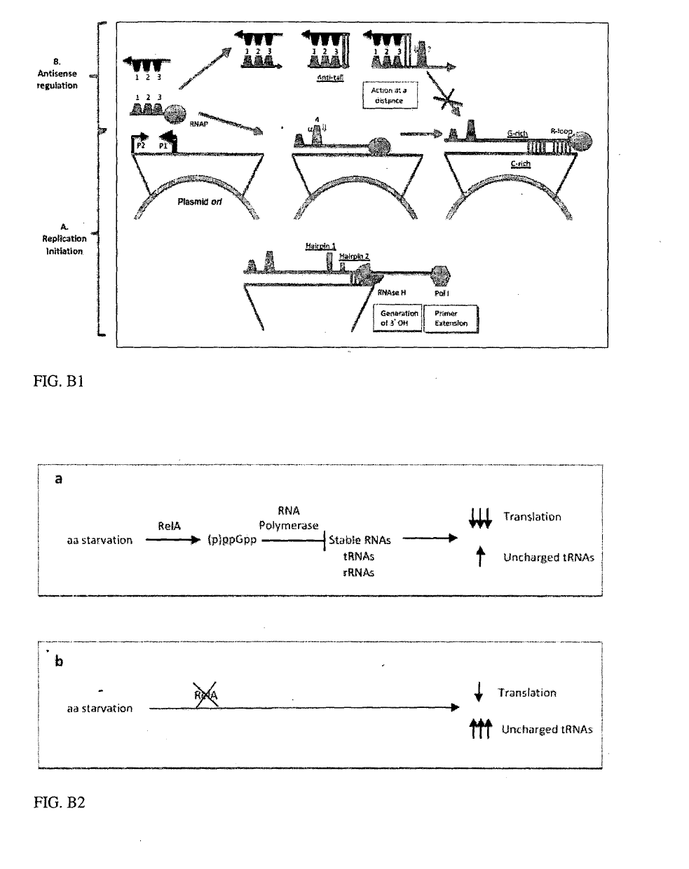

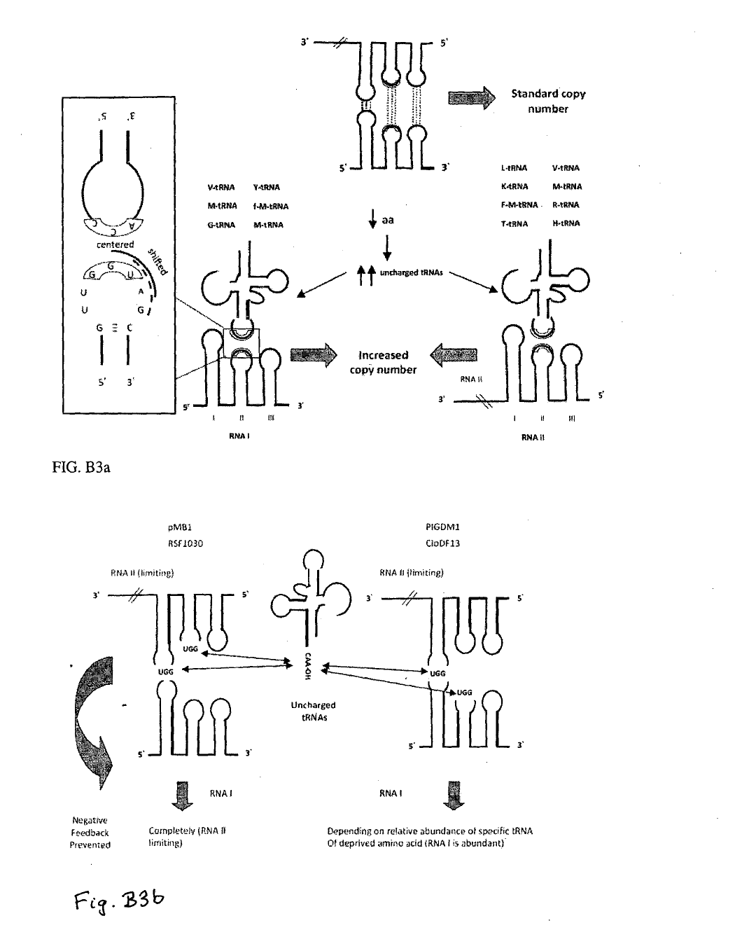

Supp. FIG. 2: Distribution of putative stabilizing mutations. 24 positively-selected ori mutations previously described inl are shown on the X axis by nucleotide position relative to the ori start. The frequency of these mutations was increased 22-fold following selection. Mutations were on average 23 nucleotides apart. The Y axis represents the number of mutant positions found within a 10 nucleotide interval starting at the position denoted on the X axis. This representation allows the identification of areas of particularly high mutation density. Clusters of high mutation density are listed, with their mutant positions, at the top. Recognizable functional features of the ColE1 plasmid ori with stabilizing mutations are listed at the bottom. For a comprehensive list of functional features, see Table 1.

Table B1 Sequence of ABH2 clones identified following methylating agent selection. Clones containing mutations in ori and 5′ of ori characterized further are highlighted in bold.

FIG. B1 ColE1 plasmid homeostasis. a Replication initiation. The plasmid ori encodes an RNA pre-primer that forms a stable hybrid with a 30-nucleotide stretch of the DNA template strand. This stable hybrid, known as R-loop, is processed by RNAseH to create a 3′ -OH end. Extension of this end by DNA polymerase I initiates leader strand DNA synthesis. b regulation Replication is regulated by transcription of a 108 nt-long antisense RNA encoded by the plasmid (known as RNA I), which is transcribed from antisense promoter P1. The pre-primer (known as RNA II) is transcribed from a sense promoter, P2. The antisense promoter is much stronger, resulting in a 100-fold excess of inhibitor relative to pre-primer. This constitutes a negative feedback loop, as the levels of inhibitor are proportional to the number of plasmids. The result is a specific copy number/per cell for a given set of conditions. Plasmid copy number regulation, however, is very dynamic and responds to metabolic fluctuations and to the presence of additional plasmids in the host. The short half-life of RNA I (only 2 minutes during exponential growth) facilitates a rapid response to environmental input. One unique feature about ColE1 antisense regulation is that the sequence of the inhibitor RNA overlaps with the 5′ end sequence of its target, the pre-primer RNA. Thus, the inhibitor is complementary to its target and hybridizes with it. The inhibitor-target interaction is determined by the formation of three stem-loops that leave 6 to 7 unpaired residues at the tip. These loops, known as stem-loops 1, 2 and 3 (SL1, SL2 and SL3) form in both target and inhibitor RNAs and mediate their initial interaction. Next, the 5′ end of the inhibitor (known as antitail) nucleates the hybridization between the two RNAs to form an RNAI-RNAII duplex. Transcription of the pre-primer past the initial 200 nucleotides leads to the formation of a new loop (SL4), through the interaction of two sequence domains, a and b. Once SL4 is formed, the pre-primer transcript can no longer bind the antisense inhibitor. Thus, although the inhibitor RNA is present in vast excess, it has a short window of action because it is dependent on the kinetics of folding of its target.

FIG. B2 Metabolic response to amino acid starvation (modified from (7)) Stringent response. Amino acid depletion causes ribosome idling, which is sensed by a ribosomal protein, ppGpp-synthetase I (encoded by relA). Rel A activation produces an alarmon, (p)ppGpp, which interacts with RNA Polymerase. Conformational changes in the RNA polymerase lead to changes in promoter specificity, reducing the synthesis of stable RNAs and increasing expression of biosynthetic pathways (reviewed in (7)). This leads to a marked suppression in protein synthesis. b Relaxed response. RelA strains, which are defective in ppGpp synthetase I, maintain low levels of (p)ppGpp under conditions of amino acid starvation. This maintains the level of protein synthesis, but generates high levels of uncharged tRNAs, which eventually also have an impact on levels of translation; (modified from (7)).

FIG. B3 Antisense RNA disregulation by recombinant gene expression: models of interference by uncharged tRNAs tRNAs in relA stains a Formation of codon-anticodon complexes with tRNA. A structural similarity and >40% sequence homology was noticed between SL1, 2, and 3 of RNA I, II or both and the cloverleaf structure of t-RNAs. Yavachev et al. postulated that competitive hybridization between the anticodon loop of tRNA and the corresponding anticodon-like loops of RNA I or RNA II could interfere with the formation of RNAI/RNAII hybrids. This model predicts that changes in plasmid copy number with deprivation of individual amino acids should correlate with the homology between the corresponding tRNAs and loops in RNA I and II. Note that each unpaired loop provides three different options for hybridization with tRNA anticodon sequences. A 7-nt loop is shown in the inset as an example: centered (boxed), shifted (continuous line) and very shifted (broken line). Based on solvent exposure, centered interactions are assumed to be the preferred ones, while the very shifted ones would be the least preferred ones. (b). CAA-OH tRNA hybridization with UGG sequence of RNAI or RNA II. 3′-CAA of uncharged tRNAs hybridizes with the GGU motif of either RNA I or RNAII (depending on the specific sequence of each ori), and this bond is stabilized because a proton given by the CCA is trapped by an electron hole (GG+) at RNA I, RNA II loops (8). The specific effects of individual amino acid deprivation are explained because in ColE1 plasmids, the GGU . . . G sequence is encoded in RNA I, which is the more abundant of the two ori transcripts and therefore sensitive to the levels of uncharged tRNA, which in turn corresponds to the relative abundance of different amino acids in E. coli proteins. This model predicts that starvation for any amino acid should lead to runaway plasmid replication in the case of PIGDM1, CIoDF13 and other members of the ColE1/pMB1 family of enzymes that encode the GGU . . . G in the RNAII transcript.

FIG. B4. Engineered, highly-stable ColE1 plasmid origins of replication. Cartoon showing a 269nt duplication at the 5′ end of ori, bearing SL1-4, which are stem-loop structures mediating antisense RNA regulation of plasmid replication initiation. In addition, ori mutations modulating plasmid copy number were cloned into the original (i.e. not duplicated) ori sequence, using an engineered ClaI. Mutations located within the 269 nt area of overlap between the duplicated segment and the ori are highlighted in bold, as these could lead SL1-4 to function as an additional negative feedback loop. For simplicity, mutations located downstream of the RNA/DNA switch are not shown.

FIG. B5 Effects of modified oris on copy number. Band intensity for the relevant plasmid is presented for each of the characterized mutants, compared to the intensity of the ori pGFPuv(pMBLI).

FIG. B6. Effects of modified oris on plasmid stability. Two control plasmids (pGFPuv and pGFPuv with the 269nt duplication) and eight mutants were characterized forplasmid retention. BL21 (WT E. coli K) cells were transformed with each of the listed constructs and grown either without or with carbenicillin selection. After reaching saturation each culture was diluted to an equivalent of OD 0.001 and 10 μl were passed into 4 ml of LB with or without antibiotic. Each passage represented 40,000× amplification or 15 generations. The number of cells retaining antibiotic resistance following 3, 8 or 13 passages in the absence of antibiotic is shown as % relative to cells grown in the presence of antibiotic.

FIG. B7: Plasmid copy number distribution in the population. Transformants were grown in the presence of antibiotic and passed once in the presence (FIG. 7a) or absence (FIG. 7b) of antibiotic. Our constructs carried a GFP fluorescent marker. The GFP fluorescence of individual cells was measured using a FACS machine as a readout for plasmid copy number.

### GENERAL REPRESENTATIONS CONCERNING THE DISCLOSURE

All papers, applications and references disclosed are hereby incorporated by reference for all purposes.

Origins or Replication (‘Oris’) are numbered according to the following reference: ‘Nucleotide sequence of the region required for maintenance of colicin E1 plasmid.'by Ohmori H, Tomizawa J., Mol Gen Genet. 1979 Oct. 3; 176(2):161-70, with ‘0’ representing the start of primer transcription.

As used in this specification, the singular forms “a”, “an”, and “the” include plural reference unless the context clearly dictates otherwise. Where reference is made in this specification to a method comprising two or more defined steps, the defined steps can be carried out in any order or simultaneously (except where the context excludes that possibility).

The term “antisense” refers to any composition containing a nucleic acid sequence which is complementary to the “sense” strand of a specific nucleic acid sequence. Antisense molecules may be produced by any method including synthesis or transcription. Once introduced into a cell, the complementary nucleotides combine with natural sequences produced by the cell to form duplexes and to block either transcription or translation. The designation “negative” or “minus” can refer to the antisense strand, and the designation “positive” or “plus” can refer to the sense strand.

A “fragment” is a unique portion of a parent sequence which is identical in sequence to but shorter in length than the parent sequence. A fragment may comprise up to the entire length of the defined sequence, minus one nucleotide/amino acid residue. For example, a fragment may be at least 5, 10, 15, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50% of a polypeptide) as shown in a certain defined sequence. Clearly these lengths are exemplary, and any length that is supported by the specification, including the Sequence Listing, tables, and figures, may be encompassed by the present embodiments.

The phrases “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity between polynucleotide sequences may be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window=4, and “diagonals saved”=4. The “weighted” residue weight table is selected as the default. Percent identity is reported by CLUSTAL V as the “percent similarity” between aligned polynucleotide sequence pairs. Alternatively, a suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403-410). The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.9 (May 7, 1999) set at default parameters. Such default parameters may be, for example: Matrix: BLOSUM62; Reward for match: 1; Penalty for mismatch: −2; Open Gap: 5 and Extension Gap: 2 penalties; Gap x drop-off: 50; Expect: 10; Word Size: 11; Filter: on. Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 7, 1999) set at default parameters. Such a pair of nucleic acids may show, for example, at least 50%, at least 60%, at least 70%, at least 80%, at least 85%, at least 90%, at least 95% or at least 98% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotide sequences that vary from one species to another. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

Plasmid stability is a function of the degree to which a plasmid is retained in a population through successive generations. In the present disclosure plasmid stability was tested by transforming cells with a plasmid and passaging the transformed cells in the absence of antibiotic and plating them in the presence of antibiotic to determine the number of cells still retaining the plasmid.

### DETAILED DESCRIPTION OF THE INVENTION

We report the generation of ColE1 plasmid origins of replication containing duplicated antisense feedback loop sequence and additional point mutations that exhibit high stability in the absence of drug selection. Super-stable plasmids show a narrowed range of copy numbers, suggesting that copy number distribution is a key determinant of plasmid stability and that it is susceptible to genetic modulation. Our engineered plasmids should improve yields of large-scale recombinant gene expression.

Certain methods and engineered sequences important to the invention are disclosed in Recent Patents on DNA & Gene Sequences 2010, 4, 58-73; Bentham Science Publishers Ltd; Modulation of ColE1-Like Plasmid Replication for Recombinant Gene Expression; Manel Camps, which is hereby incorporated by reference for all purposes.

ColE1-like plasmid origins of replication control the replication of a variety of popular shuttle and expression vectors. Transcription of the plasmid origin of replication (ori) generates an RNA primer that is extended by DNA polymerase I (pol I) following the formation of a stable RNA-DNA hybrid (R-loop) at the 3′ end. ColE1 plasmid copy number (dosage) is controlled at the level of replication initiation by an antisense RNA feedback loop. The antisense RNA is generated by transcription of 5′ terminal ori sequence in the 3′ to 5′ direction. Hybridization between primer and antisense RNAs blocks replication initiation further downstream by action at a distance.

Plasmids represent a burden for the host cell and are lost in the absence of a functional selection. The rate of plasmid loss is determined in part by the relative selective advantage of plasmid-free cells, which is inversely proportional to the metabolic burden of the recombinant construct. We used pGFPuv (Clontech), a ColE1 plasmid bearing “cycle 3” GFP and carbenicillin as a selectable marker, to study the dynamics of plasmid loss in the absence of antibiotic marker selection. Since GFP fluorescence is proportional to plasmid copy number we can monitor plasmid loss by flow cytometry (Table 1a and FIG. 1) (See Million-Weaver, S., Alexander, D. L., Allen, J. M. & Camps, M. Regulation of plasmid copy number: evidence of a critical role for the evolution of new biochemical activities. IN: Microbial Metabolic Engineering: Methods and Protocols Methods in Molecular Biology, In press, 2011. Levels of fluorescence intensity in individual cells allow us to distinguish dark (plasmid-less) cells from dim (low plasmid copy number) cells and bright (high plasmid copy number) cells (Table 1a and FIG. 1a-d). Here we report the generation of ColE1 plasmid origins of replication that are highly stable in the absence of a functional selection, which should improve yields for recombinant gene expression in situations where functional selections are impractical.

In the presence of carbenicillin, plasmid copy number in individual cells follows a one-sided distribution, with a maximum and a “tail” of cells with low plasmid dosage (FIG. 1a). This distribution profile indicates that a significant subpopulation (˜11% of the cells) maintains ColE1 plasmids at low copy number even in the presence of carbenicillin. Growth in the absence of carbenicillin predictably results in the rapid loss of plasmid, as seen both by flow cytometry (Table 1a and FIG. 1a) and by monitoring retention of the carbenicillin resistance marker (Table 1b and FIG. 1e). The low copy number subpopulation detected in the presence of antibiotic likely facilitates the rapid plasmid loss once the antibiotic is removed, as these cells will lose plasmid stochastically during segregation, and plasmid deficient cells can then expand in the absence of drug.

Key elements for antisense regulation are contiguous within the 5′-terminal sequence of the ColE1 ori. We reasoned that by duplicating this sequence we might alter the distribution of plasmid dosage in the population and thus alter plasmid stability. We created a plasmid ori with a Duplicated Antisense Sequence (DAS) by duplicating the 5′-terminal 269 nt of ori sequence. This construct has two identical, contiguous sequences bearing antisense regulatory elements (Suppl. FIG. 1a, cartoon representation; Suppl. FIG. 1b, corresponding annotated sequence). Flow cytometry analysis shows that in the presence of carbenicillin DAS plasmids exhibit decreased plasmid copy number variation relative to the wild-type pGFPuv plasmid (FIG. 1b). Low copy DAS cells represent only 2.4% of the total, compared to 11% for the wild-type (Table 1a). When drug selection is removed, the DAS construct is more stable than the wild-type both by flow cytometry (FIG. 1b) and by marker retention (Table 1b and FIG. 1e). By flow cytometry, the DAS construct shows an increase in low-plasmid copy cells (15%) but plasmidless cells represent a fraction of the total (4%), compared to 99% for the wild-type plasmid. Consistent with these results, we also observed a 7000-fold increase in DAS plasmid retention relative to WT ori following three passages in the absence of drug (Table 1b). The observed increased stability of DAS plasmids is consistent with a critical role of the low-plasmid copy number subpopulation in driving plasmid loss and suggests that plasmid copy number distribution can be genetically tuned.

We increased the stability of DAS plasmids further through the incorporation of stabilizing point mutations. These plasmid ori mutations (listed in Suppl. Table 1) were identified in our laboratory as undergoing strong positive selection during functional selection of a random mutant plasmid library bearing a human gene. We reasoned that the plasmid ori mutations could be increasing plasmid stability in the face of the high burden caused by expression of this mildly toxic exogenous gene. We cloned eight mutant plasmid origins of replication (listed in Table Ib) into the Cla I/Pci l sites of DAS ori construct (Suppl. FIG. 1) to determine their effect on plasmid stability. All eight mutants increased plasmid retention in the absence of carbenicillin (Table 1b). Two mutants stood out for their dramatic effects on stability: DAS-T170C G325A T479A and DAS-G44A G258A. Following 13 passages in the absence of drug, these two mutants show levels of plasmid retention of around 10⁷ per 10⁹ viable cells, compared to less than 2 in 10⁹ cells for WT pGFPuv (Table Ib). Flow cytometry also points to these two mutants as being exceptionally stable, with low percentages of low plasmid copy number cells even after 4 passages in the absence of carbenicillin (Table 1a).

To narrow the search for the mechanism by which these point mutations provide increased plasmid stability, we mapped all positively-selected ori mutations identified in our original selection to recognizable sequence elements, listed in Suppl. Table 1. See Camps, M. Modulation of ColE1-like plasmid replication for recombinant gene expression. Recent Pat DNA Gene Seq 4, 58-73 (2010) and Cesareni, G., Helmer-Citterich, M. & Castagnoli, L. Control of ColE1 plasmid replication by antisense RNA. Trends Genet 7, 230-235 (1991).

These elements include: three stem-loops (SL1-3) that initiate the formation of the primer-antisense hybrid; a stem-loop (SL-4), which makes the primer transcript refractory to antisense RNA inhibition; three stretches of sequence (α, β, γ-stems) involved in the action at a distance of the antisense RNA; one G- and one C-rich sequence stretch, guiding the formation of the R loop; and two hairpins. We looked for hotspots of positive selection based on mutation density (Suppl. FIG. 2). We found that SL1, SL2, SL4, antisense promoter and the β stem (areas involved in antisense regulation) are enriched in stabilizing mutations. We also found mutations in Hairpin 2 and in the C-rich stretch, two areas facilitating R-loop formation. Positions 325-328, and positions 477-479 are also of likely significance based on mutation density, although their function is unknown. Overall, these data indicate that plasmid stability can be fine-tuned by multiple sequence elements.

In sum, we show that the distribution of plasmid copy numbers in individual cells has an impact on plasmid retention and present two genetic approaches to enhance plasmid stability. Combined, these genetic modifications allow retention of ColE1 plasmids in the absence of drug selection to an unprecedented degree. Our super-stable constructs should be of use to increase the yield of recombinant gene expression in situations when selections are not feasible or practical, such as large-scale production.

Supplementary Materials

Supplementary Methods

Top 10 cells were transformed with either pGFPuv plasmid (WT), the DAS pGFPuv construct (DAS), or with selected DAS mutants. Cells were expanded in the presence of carbenicillin (passage 0), and a 1:105 dilution was serially passaged in 4 ml LB at 37° C. in the absence any carbenicillin (passages 1-13). Flow cytometry and β-lactamase marker retention experiments were performed independently.

Flow cytometry analysis. Cultures were washed into sheath buffer and analyzed with a Cytopeia Influx cytometer for GFP fluorescence using 200 mW Coherent 488 laser, 531/40 PMT. Viable cells were gated based upon PI exclusion. Cells were considered to have no plasmid when their fluorescence intensity was <1 and to have low plasmid dosage when their fluorescence intensity was greater than 1 but below the inflection point of the population distribution.

β-lactamase marker retention. Cells were expanded in the presence of carbenicillin (passage 0), and a 1:105 dilution was serially passaged overnight in 4 ml of LB at 37° C. in the absence of carbenicillin selection (passages 1-13). To determine the fraction of plasmids lost at specific passages, cells were plated in the presence or absence of carbenicillin and the number of CFUs was counted. Results are expressed as percentage of cells retaining carbenicillin resistance.

The sequence of Supp. FIG. 1 (b) is rewritten for the sake of clarity, below:

| 5′DAS | |
|---|---|
| GTAGAAAAGATCAAAGGATCT [symbol] [symbol] [symbol] [symbol] [symbol] TG | 60 |
| CAAACAAAAAAA [symbol] [symbol] [symbol] [symbol] CCGGATCAAGAGCTACCAACTCT | 120 |
| TTTTCCGAAGGTAACTGGCTTCAGCAGAGCGCAGATACCAAATACTGTTCTTCTAGTGTA | 180 |
| GCCGTAGTTAGGCCACCACTTCAAGAACTCTGTAGCACCGCCTACATACCTCGCTCTGCT | 240 |
| ClaI 3′DAS | |
| AATCCTGTTACCAGTGGCTGCTGCCA [symbol] CCCGTAGAAAAGATCAAAGGATCTTCTT | 300 |
| 44A 54A | |
| GAGATCCTTTTTTTCTGCGCGTAATCTGCTGCTTGCAAACAAAAAAACCACCGCTACCAG | 360 |
| 95T | |
| CGGTGGTTTGTTTGCCGGATCAAGAGCTACCAACTCTTTTTCCGAAGGTAACTGGCTTCA | 420 |
| 170C 180A 202C | |
| GCAGAGCGCAGATACCAAATACTGTTCTTCTAGTGTAGCCGTAGTTAGGCCACCACTTCA | 480 |
| 238T 238T | |
| AGAACTCTGTAGCACCGCCTACATACCTCGCTCTGCTAATCCTGTTACCAGTGGCTGCTG | 540 |
| 325A | |
| CCAGTGGCGATAAGTCGTGTCTTACCGGGTTGGACTCAAGACGATAGTTACCGGATAAGG | 600 |
| 373A | |
| CGCAGCGGTCGGGCTGAACGGGGGGTTCGTGCACACAGCCCAGCTTGGAGCGAACGACCT | 660 |
| ACACCGAACTGAGATACCTACAGCGTGAGCTATGAGAAAGCGCCACGCTTCCCGAAGGGA | 720 |
| GAAAGGCGGACAGGTATCCGGTAAGCGGCAGGGTCGGAACAGGAGAGCGCACGAGGGAGC | 780 |
| 512A | |
| TTCCAGGGGGAAACGCCTGGTATCTTTATAGTCCTGTCGGGTTTCGCCACCTCTGACTTG | 840 |
| 589C | |
| AGCGTCGATTTTTGTGATGCTCGTCAGGGGGGCGGAGCCTATGGAAAAACGCCAGCAACC | 900 |
| 643T 646T PciI | |
| GGCCTTTTTACGGTTCCTGGCCTTTTGCTGGCCTTTTGCTCACATGT | 947 |

Bold = duplicated sequence
Underline = Site of stabilizing point mutation
Italics = unique restriction site used for cloning

### Experimental Validation

Some of the below information is duplicative but is included to provide full disclosure of the invention. The following describes tests done to determine the effects of duplication on various aspects of plasmid replication and maintenance. We detected an impact on plasmid copy number, and a strong effect on plasmid stability.

Plasmid copy number: The duplication of the 269nt section of sequence bearing SL1-4 increased plasmid copy number by 3-fold (FIG. B5). This duplicated segment, however, did not add to (and in some cases even decreased) the effect of our ori point mutations on plasmid copy number. Thus, our constructs including both the duplication and the point mutations show plasmid copy numbers ranging between ˜1 and 2.4-fold over wildtype, whereas the point mutations by themselves caused between 0.9 and 3.9-fold increases in plasmid copy number (Table B1 and FIG. B5).

Plasmid stability: We tested the effect of the duplication on plasmid stability by passing transformed cells in the absence of antibiotic and plating them in the presence of antibiotic to determine the number of cells still retaining the plasmid. Each passage represents ˜15 generations or a 40,000-fold amplification in cell number. We found that the SL1-4 duplication resulted in a substantial increase in stability following 3, 8, and 13 passages (FIG. B6). Our plasmid copy number mutants, when introduced into the ori (leaving wild-type sequence in the duplicated section) enhanced this effect to different degrees. This is shown in FIG. B6, with controls as circles, triangles for mutants with a moderate effect, and diamonds for the two mutants with the strongest effect: T170C G325A T479A and G44A G258A. These two mutants clearly stood out, showing close to 1% plasmid retention following ˜200 generations of passage in the absence of selection. Thus, these two mutant oris allow stable replication of plasmid-encoded genes for multiple generations without the need for selection or functional complementation. This should greatly facilitate large-scale production of DNA, proteins, and secondary metabolites.

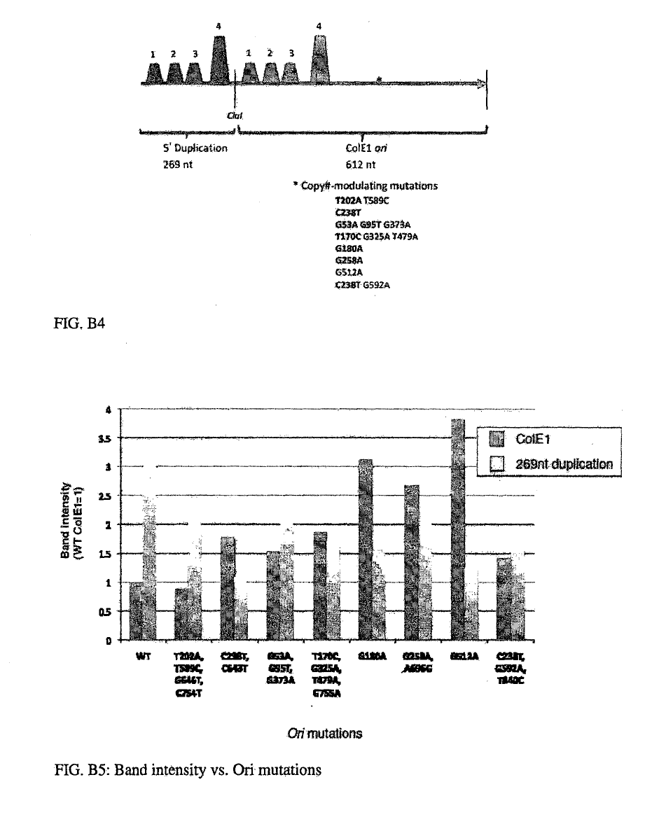

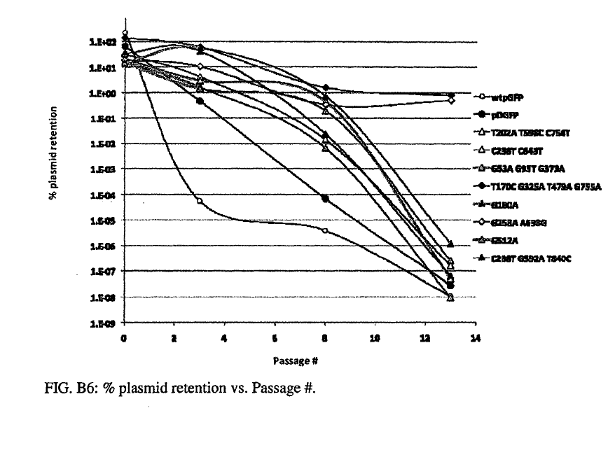

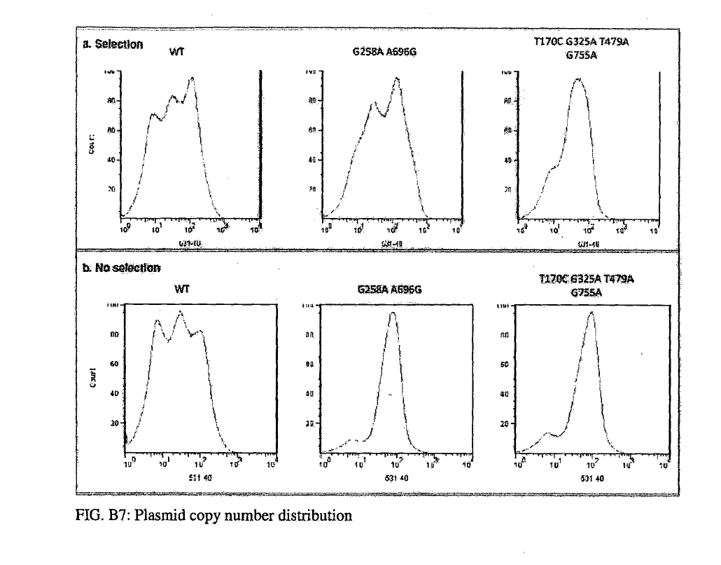

Given that the differences in copy number between the SL1-4-duplicated and their parental mutants are small and inconsistent between clones, these copy number is unlikely a mechanism significantly contributing to the observed enhancement in plasmid stability. Instead is likely attributable to increased efficiency of replication initiation regulation. We previously showed that measurement of GFP fluorescence of a plasmidborne GFP gene can be used to determine plasmid copy number. Measuring plasmid GFP fluorescence in individual cells by FACS sorting confirms the presence of a more uniform distribution of plasmid copy numbers in our two constructs with high stability relative to wild-type (FIG. B7). This is especially true in the absence of drug (FIG. B7a and B7b, respectively). This decreased variability in plasmid copy number explains the observed increase in stability by reducing the frequency of random plasmid loss. The observed enhancement in plasmid copy number regulation is likely attributable to the duplication of SL1-4 (FIG. B4, B6). However, the presence of mutations modulating plasmid copy number improves plasmid stability, particularly two mutants, namely T170C G325A T479A and G44A G258A (FIG. B6). We ignore at present how these point mutations contribute to plasmid stability. Six out of seven of the mutants bear mutations within the 269 nt duplicated sequence (FIG. 4 in bold). The observed sequence drift in one of the SL1-4 copies could facilitate its role as an additional negative feedback loop. We also noted the presence of two GOA mutations in the vicinity of the g-stem (G325A and 0258A) in our two most stable clones. The g stem is involved in plasmid copy number regulation by hybridizing with another sequence domain (b) to mediate the action at a distance of antisense RNA blocking plasmid replication initiation. Our observation suggests that the sequence context surrounding the g stem domain may be of relevance for plasmid stability.

1. An engineered plasmid whose replication is stable over multiple generations in the absence of any genetic or chemical selection, wherein the plasmid comprises a Col E1 origin of replication containing a duplicated antisense feedback loop sequence and at least one additional stabilizing point mutation.

2. The plasmid of claim 1 whose replication is stable over multiple generations in the absence of any genetic or chemical selection, comprising an engineered ColE1 sequence or variant thereof, wherein the ColE1 sequence includes at least one origin of replication comprising one or more duplicated sequences selected from the group consisting of:
(a) a duplication of the antisense feedback loops 1, 2, and 3 having at least 80% sequence identity with the wild type sequence,
AAAAAACCACCGCTACCAGCGGTGGTTTGTTTGCCGGATCAAGAGCTACCAA CTCTTTTTCCGAAGGTAACTGGCTTCAGCAGAG ,
(b) a duplication of the P2 promoter sequence having at least 80% sequence identity with the wild type sequence,
TCTTGAGATCCTTTTTTTCTGCGCGTAATCTGCTGCT, and
(c) a duplication of the Stem-Loop 4 sequence having at least 80% sequence identity with the wild type sequence,
AAATACTGTTCTTCTAGTGTAGCCGTAGTTAGGCCACCACTTCA
(d) a duplication of the P1 antisense RNA promoter sequence having at least 80% sequence identity with the wild type sequence
ACCAAATACTGTTCTTCTAGTGTAGCCGTAGTTAGGCCACCACT
(e) a duplication of the beta-stem sequence having at least 80% sequence identity with the wild type sequence TCGCTCTGCTAATCCTGTTACC.

3. The plasmid of claim 2 wherein the named duplicated sequence(s) have at least 90% sequence identity with the wild type sequence.

4. The plasmid of claim 2 wherein the named duplicated sequence(s) have at least 95% sequence identity with the wild type sequence.

5. The plasmid of claim 2 wherein the named duplicated sequence(s) have at least 99% sequence identity with the wild type sequence.

6. The plasmid of claim 2 comprising a duplication of the entire 5′ terminal 269 nucleotides of the ori sequence,
GTAGAAAAGATCAAAGGATCTTCTTGAGATCCTTTTTTTCTGCGCGTAATCTG CTGCTTGCAAACAAAAAAACCACCGCTACCAGCGGTGGTTTGTTTG CCGGATCAAGAGCTACCAACTCTTTTTCCGAAGGTAACTGGCTTCAGCAGAG CGCAGATACCAAATACTGTTCTTCTAGTGTAGCCGTAGTTAGGCCAC CACTTCAAGAACTCTGTAGCACCGCCTACATACCTCGCTCTGCTAATCCTGTT ACCAGTGGCTGCTGCCA.

7. The plasmid of claim 2 additionally comprising at least one point mutation of a nucleotide residue selected from the group consisting of:
Position 44 in the P1 primer promoter region
Position 53 in the P1 primer promoter region
Position 95 in the SL1 region
Position 113 in the SL2 region
Position 116 in the SL2 region
Position 170 in the antisense promoter region
Position 180 in the antisense promoter (overlapping with SL4 region).
Position 189 in the antisense promoter (overlapping with SL4 region)
Position 198 in the antisense promoter (overlapping with SL4 region)
Position 202 in the antisense promoter (overlapping with SL4 region)
Position 238 in the beta-stem region
Position 512 in the Hairpin 2 region
Position 592 in the C-rich region, within area of displaced strand
Position 592 in the displaced strand region
Position 325 in the Intervening sequence region
Position 328 in the Intervening sequence region
Position 338 in the Intervening sequence region
Position 373 in the Intervening sequence region
Position 409 in the Intervening sequence region
Position 477 in the Intervening sequence region
Position 479 in the Intervening sequence region
Position 589 in the Intervening sequence region
Position 643 in the DNA extension region, and
Position 646 in the DNA extension region.

8. The plasmid of claim 2 additionally comprising the three point mutation(s) shown in DAS-C170T G325A and T479A : C to T at nucleotide position 170, G to A at nucleotide position 325, [[an]] and T to A at position 479.

9. The plasmid of claim 2 additionally comprising the two point mutation(s) shown in DAS- DAS-G44A G258A: G to A mutation at nucleotide position 44, and G to A at nucleotide position 258.

10. The plasmid of claim 2 additionally comprising point mutation(s) in an region selected from the group consisting of: SL1, SL2, SL4, antisense promoter, f3 stem, Hairpin 2, the C-rich region, the region defined by nucleotides 325-328, and the region defined by nucleotides 477-479.

11. The plasmid of claim 2 wherein, when transformed into a host cell, the plasmid exhibits levels of plasmid retention at least 100 times that of a wild type control under the same conditions following 13 passages (200 generations).

12. A method for controlling plasmid copy number by modulation of plasmid copy number variance within a population, and not controlling the controlling the average copy number, the method comprising duplication of ori regions, and production of point mutations selected from the group consisting of:
Position 44 in the P1 primer promoter region
Position 53 in the P1 primer promoter region
Position 95 in the SL1 region
Position 113 in the SL2 region
Position 116 in the SL2 region
Position 170 in the antisense promoter region
Position 180 in the antisense promoter (overlapping with SL4 region).
Position 189 in the antisense promoter (overlapping with SL4 region)
Position 198 in the antisense promoter (overlapping with SL4 region)
Position 202 in the antisense promoter (overlapping with SL4 region)
Position 238 in the beta-stem region
Position 512 in the Hairpin 2 region
Position 592 in the C-rich region, within area of displaced strand
Position 592 in the displaced strand region
Position 325 in the Intervening sequence region
Position 328 in the Intervening sequence region
Position 338 in the Intervening sequence region
Position 373 in the Intervening sequence region
Position 409 in the Intervening sequence region
Position 477 in the Intervening sequence region
Position 479 in the Intervening sequence region
Position 589 in the Intervening sequence region
Position 643 in the DNA extension region, and
Position 646 in the DNA extension region.

13. A method for production of polynucleotides, proteins, glycoproteins, or secondary metabolites, the method comprising culturing a cell transformed with an engineered expression vector providing stable replication of plasmid-encoded genes for multiple generations without the need for selection and/or functional complementation, wherein the engineered plasmid comprises an origin of replication containing a duplicated antisense feedback loop sequence.

14. The method of claim 13 wherein the engineered expression vector is a Col E1 plasmid.

15. The method of claim 13 wherein the engineered expression vector further comprises at least one stabilizing point mutation.

16. The method of claim 13 wherein the engineered expression vector comprises one or more duplications selected from the group consisting of: (a) a duplication of the antisense feedback loop having at least 80% sequence identity with the wild type sequence, (b) a duplication of a promoter sequence having at least 80% sequence identity with the wild type sequence, (c) a duplication of a Stem-Loop sequence having at least 80% sequence identity with the wild type sequence, (d) a duplication of an antisense RNA promoter sequence having at least 80% sequence identity with the wild type sequence, and/or (e) a duplication of a beta-stem sequence having at least 80% sequence identity with the wild type sequence.

17. The method of claim 16 wherein the engineered expression vector further comprises at least one stabilizing point mutation.

18. The method of claim 17 wherein the engineered expression vector further comprises at least one or more stabilizing point mutations in an region selected from the group consisting of: SL1, SL2, SL4, antisense promoter, f3 stem, Hairpin 2, the C-rich region, the region defined by nucleotides 325-328, and the region defined by nucleotides 477-479.
